(19) United States
(12) Patent Application Publication
(10) Pub. No.: US 2023/0131334 A1
(43) Pub. Date: Apr. 27, 2023

(54) COMPANION DIAGNOSTIC BIOMARKERS OF EGFR INHIBITOR

(71) Applicants: SYNTEKABIO, INC., Daejeon (KR); KOREA FOOD & DRUG ADMINISTRATION, Cheongju-si Chungcheongbuk-do (KR)

(72) Inventors: Jong Sun JUNG, Daejeon (KR); Jong Gu LEE, Cheongju-si Chungcheongbuk-do (KR)

(73) Assignees: SYNTEKABIO, INC., Daejeon (KR); KOREA FOOD & DRUG ADMINISTRATION, Cheongju-si Chungcheongbuk-do (KR)

(21) Appl. No.: 18/068,517

(22) Filed: Dec. 19, 2022

Related U.S. Application Data
(63) Continuation-in-part of application No. PCT/KR2021/007706, filed on Jun. 18, 2021.

(30) Foreign Application Priority Data
Jun. 19, 2020 (KR) 10-2020-0075057

(51) Int. Cl. C12Q 1/6886

(52) U.S. Cl. CPC C12Q 1/6886; C12Q 2600/106; C12Q 2600/156; C12Q 2600/158

(57) ABSTRACT

The present disclosure relates to methods of predicting treatment sensitivity or drug resistance, especially for epidermal growth factor receptor (EGFR) inhibitors using leucine proline-enriched proteoglycan 1 (LEPRE1) gene expression level before or during a treatment, methods of discovering companion diagnostic biomarkers using efficacies of EGFR inhibitors on the expression of genes, including genes involved with regulation of extracellular matrix environment, or metabolism of collagen, and methods of predicting treatment sensitivity or resistance of drugs using the companion diagnostic biomarkers thereof.

### CROSS REFERENCE TO RELATED APPLICATIONS

This application is a continuation-in-part of PCT/KR2021/007706, filed Jun. 18, 2021 which claims the benefit of priority from Korean Patent Application No. 10-2020-0075057, filed Jun. 19, 2020, the contents of each of which are incorporated herein by reference.

### FIELD OF THE INVENTION

The present disclosure relates to methods of predicting treatment sensitivity or drug resistance, especially for epidermal growth factor receptor (EGFR) inhibitors using leucine proline-enriched proteoglycan 1 (LEPRE1 ) gene expression level before or during a treatment, methods of discovering companion diagnostic biomarkers using efficacies of EGFR inhibitors on the expression of genes, including genes involved with regulation of extracellular matrix environment, or metabolism of collagen, and methods of predicting treatment or drug sensitivity or resistance using the companion diagnostic biomarkers thereof.

### BACKGROUND OF THE INVENTION

With recent innovations in the next-generation sequencing (NGS) technology, gene sequencing information and expression information required to understand complex and various cancers have been rapidly secured. In addition, a catalog of somatic mutations in various cancer types and a comprehensive cancer driver mutation database were established through the formation of an international consortium. Due to these achievements, expectations for the discovery of biomarkers capable of confirming the difference between patients with each allogeneic tumor, as well as the condition of tumors, and personalized cancer treatment using the same are also rapidly increasing. However, biomarkers approved and used in clinical practice are still insufficient. Genomics of drug sensitivity in cancer (GDSC), which is a database including experimental results of drug toxicity information of 1,070 human cancer cells for 265 anticancer compounds, was published through several collaborative consortiums for integrating molecular profiling data of cancer cell lines and drug toxicity data (www.lincsproject.org), and, as a result of carrying out genetic biomarker labeling scan (GBLscan) of the present applicant by using GDSC, the correlation between LEPRE1 and drug sensitivity, which is shown when treated with an EGFR inhibitor, was confirmed.

LEPRE1 (P3H1), also known as Leprecan, is a protein belonging to the collagen prolyl hydroxylase family, has the function of hydroxylating proline of collagen constituting fibrils, and is an enzyme having an essential function for collagen synthesis and structural formation. 28 types of collagen in the human body can be largely classified depending on the function thereof into: fibrillar collagens such as type I, II, III, V, XI, XXIV, and XXVII collagens; and structural network-forming collagens such as type IV and VIII collagens. LEPRE1 plays an important role in constituting the extracellular matrix existing in the space between cells by hydroxylating the 896^{th} proline (Pro896) of type I collagen from among the above collagen types to induce the modification of collagen protein, and it has been reported that mutations in LEPRE1 induce diseases such as osteochondrodysplasia (including osteogenesis imperfecta), kyphosis, and rhizomelia

The hydroxylation of type I collagen by LEPRE1 is also involved in bone cancer and cancer-related bone metastasis, and the expression level of LEPRE1 also increases in solid cancers such as pancreatic cancer, colorectal cancer, breast cancer, and lung cancer, which is closely associated with the progression of cancer. It is also known that LEPRE1 and type I collagen also affect the formation of carcinoma associated fibroblasts, and thus play an important role in cancer progression, and the amount of type I collagen increases in bone cancer, pancreatic cancer, rectal cancer, ovarian cancer, lung cancer, and the like, compared to normal tissues, thus inducing the overexpression of TGF-β, and as a result, the proliferation of cancer cells is promoted and apoptosis is reduced. In addition, it can be seen that the modification process of type I collagen by LEPRE1 plays important roles, such as: a higher growth rate of cancer cells located close to type I collagen than that of cells located not close thereto; increased metastasis-related invasiveness; and an increase in the number of circulating tumor cells, in not only an osteogenic process but also the progression processes, such as onset and metastasis, of cancer.

Epidermal growth factor receptor (EGFR) is an oncogene, and a mutation-induced increase in the expression or activity of the gene shows a high disease association in various carcinomas, including lung cancer, head and neck cancer, and the like. It has been reported that an increase in EGFR in breast cancer induces decreased LEPRE1 expression, and the possibility of the direct binding between EGFR and LEPRE1 has also been suggested. Through the above contents, it can be seen that LEPRE1, which is closely related to the onset and progression of cancer, and type I collagen, which is a target thereof, are also associated with EGFR, which can be a direct basis for the difference in the activity of an EGFR inhibitor according to the expression level of LEPRE1 according to the present disclosure.

### SUMMARY OF THE INVENTION

One embodiment of the present disclosure provides methods of predicting treatment sensitivity or drug resistance, especially for epidermal growth factor receptor (EGFR) inhibitors determining leucine proline-enriched proteoglycan 1 (LEPRE1) gene expression level before or during a treatment.

Another embodiment of the present disclosure provides methods of predicting treatment or drug sensitivity or drug resistance, especially for epidermal growth factor receptor (EGFR) inhibitors determining expression level genes that are related to regulation of extracellular matrix environment, metabolism of collagen, or mixture of thereof, before or during a treatment.

Yet another embodiment of the present disclosure provides methods of predicting treatment or drug sensitivity or resistance by determining the expression level of companion diagnostic biomarkers involved with regulation of extracellular matrix environment, metabolism of collagen, or mixture of thereof, wherein the drugs have a similar multi-target efficacies as an epidermal growth factor receptor (EGFR) inhibitors.

In one embodiment, the present disclosure provides a method of discovering a gene for determining drug sensitivity comprising:
determining a level of a gene, wherein the gene is a LEPRE1 gene, and the drug is an EGFR inhibitor drug; and
determining drug sensitivity such that the sensitivity of the cancer cell line to an EGFR inhibitor drug is high according to an overexpression level of the LEPRE1 gene, and the resistance of the cancer cell line to the EGFR inhibitor drug is high according to an underexpression level of the LEPRE1 gene.

In another embodiment, the present disclosure provide a companion diagnostic composition for determining the sensitivity to an EGFR inhibitor drug, the companion diagnostic composition comprising an agent for measuring an RNA expression level of a LEPRE1 gene or an agent for specifying a protein expression level of the LEPRE1 gene.

In a further embodiment, the present disclosure provides a method of discovering a gene for determining drug sensitivity, the method comprising:
(A) selecting a candidate gene for determining responsiveness to a target drug through cancer companion diagnostic marker scanning (GBLscan);
(B) realizing an overexpressed state of the candidate gene in a cell line targeted by the target drug;
(C) calculating responsiveness of the target drug to the targeted cell line, in the state in which the candidate gene is overexpressed, obtained in (B);
(D) realizing an underexpressed state of the candidate gene in the cell line targeted by the target drug;
(E) calculating responsiveness of the target drug to the targeted cell line, in the state in which the candidate gene is underexpressed, obtained in (D); and
(F) verifying whether or not the candidate gene is a marker for determining responsiveness to the target drug, compared with the responsiveness calculated in (C) and (E).

### BRIEF DESCRIPTION OF DRAWINGS

FIG. 1 is a graph illustrating the relationship between gene expression and drug responsiveness.

FIG. 2 illustrates the expression levels of LEPRE1 in hematological cancer cell lines (THP-1, U-937, KG-1, and HL-60), according to the present disclosure.

FIG. 3 illustrates the expression levels of EGFR in hematological cancer cell lines (THP-1, U-937, KG-1, and HL-60), according to the present disclosure.

FIG. 4 illustrates the results of inhibiting the expression of the LEPRE1 gene in a lung cancer cell line (A549) by using siRNA, according to the present disclosure.

FIG. 5 illustrates drug responsiveness results according to the overexpression and inhibited expression of the LEPRE1 gene and the expression level of the gene, in a lung cancer cell line (A549), according to the present disclosure.

FIG. 6 illustrates drug responsiveness results (first experiment) according to the overexpression and inhibited expression of the LEPRE1 gene and the expression level of the gene, in hematological cancer cell lines (KG-1 and THP-1), according to the present disclosure.

FIG. 7 illustrates drug responsiveness results (second experiment performed at a different voltage) according to the overexpression and inhibited expression of the LEPRE1 gene and the expression level of the gene, in hematological cancer cell lines (KG-1 and THP-1), according to the present disclosure.

FIG. 8 illustrates drug responsiveness results (third experiment performed at different voltage and time) according to the overexpression and inhibited expression of the LEPRE1 gene and the expression level of the gene, in hematological cancer cell lines (KG-1 and THP-1), according to the present disclosure.

FIG. 9 illustrates whether the overexpression, inhibited expression and expression level of the LEPRE1 gene according to the present disclosure affects drug responsiveness.

### DETAILED DESCRIPTION OF THE INVENTION

The present disclosure relates to methods of predicting treatment or drug sensitivity or drug resistance, especially for epidermal growth factor receptor (EGFR) inhibitors using leucine proline-enriched proteoglycan 1 (LEPRE1) gene expression level before or during a treatment, methods of discovering companion diagnostic biomarkers using efficacies of EGFR inhibitors on the expression of genes, including genes involved with regulation of extracellular matrix environment, or metabolism of collagen, and methods of predicting treatment or drug sensitivity or resistance using the companion diagnostic biomarkers thereof.

One embodiment of the present disclosure provides methods of predicting treatment sensitivity or drug resistance, especially for epidermal growth factor receptor (EGFR) inhibitors determining leucine proline-enriched proteoglycan 1 (LEPRE1) gene expression level before or during a treatment.

Another embodiment of the present disclosure provides methods of predicting treatment sensitivity or drug resistance, especially for epidermal growth factor receptor (EGFR) inhibitors determining expression level genes that are related to regulation of extracellular matrix environment, metabolism of collagen, or mixture of thereof, before or during a treatment.

Yet another embodiment of the present disclosure provides methods of predicting treatment sensitivity or resistance of drugs by determining the expression level of companion diagnostic biomarkers involved with regulation of extracellular matrix environment, metabolism of collagen, or mixture of thereof, wherein the drugs have a similar multi-target efficacies as an epidermal growth factor receptor (EGFR) inhibitors.

In the present disclosure, the correlation between LEPRE1, discovered using the unique GBLscan method, and drug sensitivity to an EGFR inhibitor was confirmed, and the present disclosure is to provide LEPRE1 as a biomarker to be used in cancer treatment through EGFR inhibitor treatment.

The common cancer drug responsiveness information and next-generation sequencing information (genomes and transcriptomes), and a biomarker gene LEPRE1 verified through the same, and the present disclosure relates to the prediction and verification of a companion diagnostic biomarker gene and mutant composition that improve cancer drug responsiveness, when a haplotype biomarker composition or gene expression information of a gene expression regulatory region is known, and in particular, to a companion diagnostic biomarker for an EGFR inhibitor.

In the present disclosure, the correlation between LEPRE1, discovered using the unique GBLscan method of the present applicant, and drug sensitivity of an EGFR inhibitor was measured, and the present disclosure provides LEPRE1 as a biomarker to be used in cancer treatment through EGFR inhibitor treatment.

The EGFR inhibitor is selected from Erlotinib (OSI-774) HCI, Gefitinib (ZD1839), Lapatinib (GW-572016) Ditosylate, Afatinib (BIBW2992), Saracatinib (AZD0530), Vandetanib (ZD6474), Neratinib (HKI-272), Canertinib (CI-1033), Lapatinib (GW-572016), AG-490 (Tyrphostin B42), CP-724714, Dacomitinib (PF-00299804), WZ4002, Sapitinib (AZD8931), CUDC-101, AG-1478 (Tyrphostin AG-1478), PD153035 HCI, Pelitinib (EKB-569), AEE788 (NVP-AEE788), AC480 (BMS-599626), AP26113-analog (ALK-IN-1), OSI-420, WZ3146, Allitinib tosylate, Rociletinib (CO-1686), Varlitinib, Icotinib (BPI-2009H), TAK-285, WHI-P154, Daphnetin, PD168393, CNX-2006, Tyrphostin 9, AG-18, O-Demethyl-Gefitinib, AST-1306, ErbB2 inhibitor, BDTX-189, Epertinib hydrochloride, JND3229, BI-4020, Tyrphostin AG-528, AG 556, Canertinib dihydrochloride, Gefitinib-based PROTAC 3, SU5214, RG 13022, TQB3804 (EGFR-IN-7), TAS6417, Pyrotinib (SHR-1258) dimaleate, PD153035, AG 494, AG 555, Theliatinib (HMPL-309), Avitinib (AC0010), Lazertinib, Gefitinib hydrochloride, Cetuximab (anti-EGFR), Lifirafenib (BGB-283), Nazartinib (EGF816), Brigatinib (AP26113), Tucatinib, Zorifertinib (AZD3759), Afatinib (BIBW2992) Dimaleate, Erlotinib (OSI-774), CL-387785 (EKI-785), Poziotinib (HM781-36B), Osimertinib (AZD9291), AZ5104, AV-412 or pharmaceutically acceptable salt thereof.

In one embodiment, the present disclosure provides a method of discovering a gene for predicting and/or determining drug sensitivity comprising:
determining the sensitivity of a cancer cell line to a drug by determining an expression level of a gene,
determining the sensitivity of the cancer cell line to the drug is high based on an overexpression level of the gene, and the resistance of the cancer call line to the EGFR inhibitor high, based on underexpression level of the gene, wherein,
the drug is preferably an EGFR inhibitor, more preferably pelitinib;
the cancer cell line is preferably any at least one selected from hematological cancer cell lines THP-1 and KG-1, and a lung cancer cell line A549; and
the gene is preferably LEPRE1 gene and expression level of the LEPRE1 gene is preferably determined high by detecting at least one alteration selected from Table 1 below,

TABLE 1

| Chromosome | Location | Reference (Ref.) | Alteration (Alt.) | Type of alteration |
|---|---|---|---|---|
| 1 | 205849911 | C | T | Substitution |
| 1 | 205849976 | G | A | Substitution |
| 4 | 68337362 | T | C | Substitution |
| 4 | 182680440 | G | C | Substitution |
| 4 | 182680513 | G | A | Substitution |
| 20 | 32983679 | C | T | Substitution |

In an example of embodiment, the gene associated to determining sensitivity to EGFR inhibitor is selected from genes in Table 2, most of which regulate the extracellular matrix environment, similar to LEPRE1, to predict or determine the drug sensitivity such that the sensitivity of the cancer cell line to the EGFR inhibitor by measuring the expression level of the genes:

TABLE 2

| No | Gene name | Relation^{#} | No | Gene name | Relation | No | Gene name | Relation |
|---|---|---|---|---|---|---|---|---|
| 1 | A2M | Highly related | 101 | LAMB1 | Highly related | 201 | ITGA10 | Related |
| 2 | ACAN | Highly related | 102 | LAM B3 | Highly related | 202 | ITGA11 | Related |
| 3 | ADAM 10 | Highly related | 103 | LAMC1 | Highly related | 203 | ITGA2 | Related |
| 4 | ADAM 15 | Highly related | 104 | LAMC2 | Highly related | 204 | ITGA2B | Related |
| 5 | ADAM 17 | Highly related | 105 | MMP1 | Highly related | 205 | ITGA3 | Related |
| 6 | ADAM8 | Highly related | 106 | MMP10 | Highly related | 206 | ITGA4 | Related |
| 7 | ADAM9 | Highly related | 107 | MMP11 | Highly related | 207 | ITGA5 | Related |
| 8 | ADAMTS1 | Highly related | 108 | MMP12 | Highly related | 208 | ITGA6 | Related |
| 9 | ADAMTS16 | Highly related | 109 | MMP13 | Highly related | 209 | ITGA7 | Related |
| 10 | ADAMTS18 | Highly related | 110 | MMP14 | Highly related | 210 | ITGA8 | Related |
| 11 | ADAMTS4 | Highly related | 111 | MMP15 | Highly related | 211 | ITGA9 | Related |
| 12 | ADAMTS5 | Highly related | 112 | MMP16 | Highly related | 212 | ITGAD | Related |
| 13 | ADAMTS8 | Highly related | 113 | MMP17 | Highly related | 213 | ITGAE | Related |
| 14 | ADAMTS9 | Highly related | 114 | MMP19 | Highly related | 214 | ITGAL | Related |
| 15 | BCAN | Highly related | 115 | MMP2 | Highly related | 215 | ITGAM | Related |
| 16 | BMP1 | Highly related | 116 | MMP20 | Highly related | 216 | ITGAV | Related |
| 17 | BSG | Highly related | 117 | MMP24 | Highly related | 217 | ITGAX | Related |
| 18 | CAPN1 | Highly related | 118 | MMP25 | Highly related | 218 | ITGB1 | Related |
| 19 | CAPN10 | Highly related | 119 | MMP3 | Highly related | 219 | ITGB2 | Related |
| 20 | CAPN11 | Highly related | 120 | MMP7 | Highly related | 220 | ITGB3 | Related |
| 21 | CAPN12 | Highly related | 121 | MMP8 | Highly related | 221 | ITGB4 | Related |
| 22 | CAPN13 | Highly related | 122 | MMP9 | Highly related | 222 | ITGB5 | Related |
| 23 | CAPN14 | Highly related | 123 | NCSTN | Highly related | 223 | ITGB6 | Related |
| 24 | CAPN15 | Highly related | 124 | NID1 | Highly related | 224 | ITGB7 | Related |
| 25 | CAPN2 | Highly related | 125 | OPTC | Highly related | 225 | ITGB8 | Related |
| 26 | CAPN3 | Highly related | 126 | PHYKPL | Highly related | 226 | JAM2 | Related |
| 27 | CAPN5 | Highly related | 127 | PLG | Highly related | 227 | JAM3 | Related |
| 28 | CAPN6 | Highly related | 128 | PRSS1 | Highly related | 228 | KDR | Related |
| 29 | CAPN7 | Highly related | 129 | PRSS2 | Highly related | 229 | LAMA1 | Related |
| 30 | CAPN8 | Highly related | 130 | PSEN1 | Highly related | 230 | LAMA2 | Related |
| 31 | CAPN9 | Highly related | 131 | SCUBE1 | Highly related | 231 | LAMA4 | Related |
| 32 | CAPNS1 | Highly related | 132 | SCUBE3 | Highly related | 232 | LAM B2 | Related |
| 33 | CAPNS2 | Highly related | 133 | SPOCK3 | Highly related | 233 | LAMC3 | Related |
| 34 | CASP3 | Highly related | 134 | SPP1 | Highly related | 234 | LOX | Related |
| 35 | CAST | Highly related | 135 | TIMP1 | Highly related | 235 | LOXL1 | Related |
| 36 | CD44 | Highly related | 136 | TIMP2 | Highly related | 236 | LOXL2 | Related |
| 37 | CDH1 | Highly related | 137 | TLL1 | Highly related | 237 | LOXL3 | Related |
| 38 | CMA1 | Highly related | 138 | TLL2 | Highly related | 238 | LOXL4 | Related |
| 39 | COL10A1 | Highly related | 139 | TMPRSS6 | Highly related | 239 | LRP4 | Related |
| 40 | COL11A1 | Highly related | 140 | TPSAB1 | Highly related | 240 | LTBP1 | Related |
| 41 | COL11A2 | Highly related | 141 | ACTN1 | Related | 241 | LTBP2 | Related |
| 42 | COL12A1 | Highly related | 142 | ADAM 12 | Related | 242 | LTBP3 | Related |
| 43 | COL13A1 | Highly related | 143 | ADAM 19 | Related | 243 | LTBP4 | Related |
| 44 | COL14A1 | Highly related | 144 | ADAMTS14 | Related | 244 | LUM | Related |
| 45 | COL15A1 | Highly related | 145 | ADAMTS2 | Related | 245 | MADCAM1 | Related |
| 46 | COL16A1 | Highly related | 146 | ADAMTS3 | Related | 246 | MATN1 | Related |
| 47 | COL17A1 | Highly related | 147 | AGRN | Related | 247 | MATN3 | Related |
| 48 | COL18A1 | Highly related | 148 | APP | Related | 248 | MATN4 | Related |
| 49 | COL19A1 | Highly related | 149 | ASPN | Related | 249 | MFAP2 | Related |
| 50 | COL1A1 | Highly related | 150 | BGN | Related | 250 | MFAP3 | Related |
| 51 | COL1A2 | Highly related | 151 | BMP10 | Related | 251 | MFAP4 | Related |
| 52 | COL23A1 | Highly related | 152 | BMP2 | Related | 252 | MFAP5 | Related |
| 53 | COL25A1 | Highly related | 153 | BMP4 | Related | 253 | MUSK | Related |
| 54 | COL26A1 | Highly related | 154 | BMP7 | Related | 254 | NCAM1 | Related |
| 55 | COL2A1 | Highly related | 155 | CASK | Related | 255 | NCAN | Related |
| 56 | COL3A1 | Highly related | 156 | CD151 | Related | 256 | NID2 | Related |
| 57 | COL4A1 | Highly related | 157 | CD47 | Related | 257 | NRXN1 | Related |
| 58 | COL4A2 | Highly related | 158 | CEACAM1 | Related | 258 | NTN4 | Related |
| 59 | COL4A3 | Highly related | 159 | CEACAM6 | Related | 259 | P3H1 | Related |
| 60 | COL4A4 | Highly related | 160 | CEACAM8 | Related | 260 | P3H2 | Related |
| 61 | COL4A5 | Highly related | 161 | COL20A1 | Related | 261 | P3H3 | Related |
| 62 | COL4A6 | Highly related | 162 | COL21A1 | Related | 262 | P4HA1 | Related |
| 63 | COL5A1 | Highly related | 163 | COL22A1 | Related | 263 | P4HA2 | Related |
| 64 | COL5A2 | Highly related | 164 | COL24A1 | Related | 264 | P4HA3 | Related |
| 65 | COL5A3 | Highly related | 165 | COL27A1 | Related | 265 | P4HB | Related |
| 66 | COL6A1 | Highly related | 166 | COL28A1 | Related | 266 | PCOLCE | Related |
| 67 | COL6A2 | Highly related | 167 | COLGALT1 | Related | 267 | PCOLCE2 | Related |
| 68 | COL6A3 | Highly related | 168 | COLGALT2 | Related | 268 | PDGFA | Related |
| 69 | COL6A5 | Highly related | 169 | COMP | Related | 269 | PDGFB | Related |
| 70 | COL6A6 | Highly related | 170 | CRTAP | Related | 270 | PECAM1 | Related |
| 71 | COL7A1 | Highly related | 171 | DAG1 | Related | 271 | PLEC | Related |
| 72 | COL8A1 | Highly related | 172 | DDR1 | Related | 272 | PLOD1 | Related |
| 73 | COL8A2 | Highly related | 173 | DDR2 | Related | 273 | PLOD2 | Related |
| 74 | COL9A1 | Highly related | 174 | DMD | Related | 274 | PLOD3 | Related |
| 75 | COL9A2 | Highly related | 175 | DMP1 | Related | 275 | PPIB | Related |
| 76 | COL9A3 | Highly related | 176 | DSPP | Related | 276 | PRKCA | Related |
| 77 | CTRB1 | Highly related | 177 | DST | Related | 277 | PTPRS | Related |
| 78 | CTRB2 | Highly related | 178 | EFEMP1 | Related | 278 | PXDN | Related |
| 79 | CTSB | Highly related | 179 | EFEMP2 | Related | 279 | SDC1 | Related |
| 80 | CTSD | Highly related | 180 | EMILIN1 | Related | 280 | SDC2 | Related |
| 81 | CTSG | Highly related | 181 | EMILIN2 | Related | 281 | SDC3 | Related |
| 82 | CTSK | Highly related | 182 | EMILIN3 | Related | 282 | SDC4 | Related |
| 83 | CTSL | Highly related | 183 | F11R | Related | 283 | SERPINE1 | Related |
| 84 | CTSS | Highly related | 184 | FBLN1 | Related | 284 | SERPINH1 | Related |
| 85 | CTSV | Highly related | 185 | FBLN2 | Related | 285 | SH3PXD2A | Related |
| 86 | DCN | Highly related | 186 | FBLN5 | Related | 286 | SPARC | Related |
| 87 | ELANE | Highly related | 187 | FGA | Related | 287 | TGFB1 | Related |
| 88 | ELN | Highly related | 188 | FGB | Related | 288 | TGFB2 | Related |
| 89 | FBN1 | Highly related | 189 | FGF2 | Related | 289 | TGFB3 | Related |
| 90 | FBN2 | Highly related | 190 | FGG | Related | 290 | THBS1 | Related |
| 91 | FBN3 | Highly related | 191 | FMOD | Related | 291 | TNC | Related |
| 92 | FN1 | Highly related | 192 | GDF5 | Related | 292 | TNN | Related |
| 93 | FURIN | Highly related | 193 | HAPLN1 | Related | 293 | TNR | Related |
| 94 | HSPG2 | Highly related | 194 | IBSP | Related | 294 | TNXB | Related |
| 95 | HTRA1 | Highly related | 195 | ICAM1 | Related | 295 | TRAPPC4 | Related |
| 96 | KLK2 | Highly related | 196 | ICAM2 | Related | 296 | TTR | Related |
| 97 | KLK7 | Highly related | 197 | ICAM3 | Related | 297 | VCAM1 | Related |
| 98 | KLKB1 | Highly related | 198 | ICAM4 | Related | 298 | VCAN | Related |
| 99 | LAMA3 | Highly related | 199 | ICAM5 | Related | 299 | VTN | Related |
| 100 | LAMA5 | Highly related | 200 | ITGA1 | Related | 300 | VWF | Related |

#Relation to regulation of extracellular matrix environment. “Highly Related” are genes with more than 5 reports of the relation, and “Related” are genes with less than 5 reports of the relation among the citations during 2000-2021.

In another example of the method, the gene associated to sensitivity to EGFR inhibitors is selected from the genes shown in Table 3, most of which regulate the metabolism of collagen, similar to LEPRE1, to predict or determine the drug sensitivity such that the sensitivity of the cancer cell line to the EGFR inhibitor by measuring the expression level of the genes:

TABLE 3

| No | Gene name | Relation^ | No | Gene name | Relation | No | Gene name | Relation |
|---|---|---|---|---|---|---|---|---|
| 1 | BMP1 | Highly related | 49 | ADAM 17 | Related | 97 | KLK8 | Related |
| 2 | COL10A1 | Highly related | 50 | ADAM9 | Related | 98 | KLK9 | Related |
| 3 | COL11A1 | Highly related | 51 | ADAMTS14 | Related | 99 | KLKB1 | Related |
| 4 | COL11A2 | Highly related | 52 | ADAMTS2 | Related | 100 | KNG1 | Related |
| 5 | COL12A1 | Highly related | 53 | ADAMTS3 | Related | 101 | LAMA3 | Related |
| 6 | COL13A1 | Highly related | 54 | CD151 | Related | 102 | LAMB3 | Related |
| 7 | COL14A1 | Highly related | 55 | COL28A1 | Related | 103 | LAMC2 | Related |
| 8 | COL15A1 | Highly related | 56 | COLGALT1 | Related | 104 | LOX | Related |
| 9 | COL16A1 | Highly related | 57 | COLGALT2 | Related | 105 | LOXL1 | Related |
| 10 | COL17A1 | Highly related | 58 | CRTAP | Related | 106 | LOXL2 | Related |
| 11 | COL18A1 | Highly related | 59 | CTSB | Related | 107 | LOXL3 | Related |
| 12 | COL19A1 | Highly related | 60 | CTSD | Related | 108 | LOXL4 | Related |
| 13 | COL1A1 | Highly related | 61 | CTSK | Related | 109 | MMP1 | Related |
| 14 | COL1A2 | Highly related | 62 | CTSL | Related | 110 | MMP10 | Related |
| 15 | COL20A1 | Highly related | 63 | CTSS | Related | 111 | MMP11 | Related |
| 16 | COL21A1 | Highly related | 64 | CTSV | Related | 112 | MMP12 | Related |
| 17 | COL22A1 | Highly related | 65 | DST | Related | 113 | MMP13 | Related |
| 18 | COL23A1 | Highly related | 66 | ELANE | Related | 114 | MMP14 | Related |
| 19 | COL24A1 | Highly related | 67 | F10 | Related | 115 | MMP15 | Related |
| 20 | COL25A1 | Highly related | 68 | F11 | Related | 116 | MMP19 | Related |
| 21 | COL26A1 | Highly related | 69 | F12 | Related | 117 | MMP2 | Related |
| 22 | COL27A1 | Highly related | 70 | F13A1 | Related | 118 | MMP20 | Related |
| 23 | COL2A1 | Highly related | 71 | F13B | Related | 119 | MMP3 | Related |
| 24 | COL3A1 | Highly related | 72 | F2 | Related | 120 | MMP7 | Related |
| 25 | COL4A1 | Highly related | 73 | F3 | Related | 121 | MMP8 | Related |
| 26 | COL4A2 | Highly related | 74 | F5 | Related | 122 | MMP9 | Related |
| 27 | COL4A3 | Highly related | 75 | F7 | Related | 123 | P3H1 | Related |
| 28 | COL4A4 | Highly related | 76 | F8 | Related | 124 | P3H2 | Related |
| 29 | COL4A5 | Highly related | 77 | F9 | Related | 125 | P3H3 | Related |
| 30 | COL4A6 | Highly related | 78 | FGA | Related | 126 | P4HA1 | Related |
| 31 | COL5A1 | Highly related | 79 | FGB | Related | 127 | P4HA2 | Related |
| 32 | COL5A2 | Highly related | 80 | FGG | Related | 128 | P4HA3 | Related |
| 33 | COL5A3 | Highly related | 81 | FURIN | Related | 129 | P4HB | Related |
| 34 | COL6A1 | Highly related | 82 | ITGA6 | Related | 130 | PCOLCE2 | Related |
| 35 | COL6A2 | Highly related | 83 | ITGB4 | Related | 131 | PHYKPL | Related |
| 36 | COL6A3 | Highly related | 84 | KLK1 | Related | 132 | PLEC | Related |
| 37 | COL6A5 | Highly related | 85 | KLK10 | Related | 133 | PLOD1 | Related |
| 38 | COL6A6 | Highly related | 86 | KLK11 | Related | 134 | PLOD2 | Related |
| 39 | COL7A1 | Highly related | 87 | KLK12 | Related | 135 | PLOD3 | Related |
| 40 | COL8A1 | Highly related | 88 | KLK13 | Related | 136 | PPIB | Related |
| 41 | COL8A2 | Highly related | 89 | KLK14 | Related | 137 | PROC | Related |
| 42 | COL9A1 | Highly related | 90 | KLK15 | Related | 138 | PROS1 | Related |
| 43 | COL9A2 | Highly related | 91 | KLK2 | Related | 139 | PRSS2 | Related |
| 44 | COL9A3 | Highly related | 92 | KLK3 | Related | 140 | PXDN | Related |
| 45 | PCOLCE | Highly related | 93 | KLK4 | Related | 141 | SERPINC1 | Related |
| 46 | TLL1 | Highly related | 94 | KLK5 | Related | 142 | SERPINH1 | Related |
| 47 | TLL2 | Highly related | 95 | KLK6 | Related | 143 | TFPI | Related |
| 48 | ADAM 10 | Related | 96 | KLK7 | Related | 144 | THBD | Related |
| | | | | | | 145 | TMPRSS6 | Related |

^Relation to regulation of metabolism of collagen. “Highly Related” are genes with more than 5 reports of the relation, and “Related” are genes with less than 5 reports of the relation among the citations during 2000-2021.

In yet another embodiment of the present disclosure provides methods to predict and/or determine sensitivity to the drug having multi-target efficacy as shown in Table 4 or an efficacy profile similar thereto, similar to EGFR inhibitor, preferably Pelitinib, by determining expression level of genes in Table 2, Table 3 or a mixture thereof:

TABLE 4

| Target Name | Standard Type | Standard Relation | Standard Value | Standard Units |
|---|---|---|---|---|
| Epidermal growth factor receptor erbB1 | Kd | ‘=’ | 0.23 | nM |
| Epidermal growth factor receptor erbB1 | Kd | ‘=’ | 0.24 | nM |
| Epidermal growth factor receptor erbB1 | Kd | ‘=’ | 0.24 | nM |
| Epidermal growth factor receptor erbB1 | Kd | ‘=’ | 0.27 | nM |
| Epidermal growth factor receptor erbB1 | Kd | ‘=’ | 0.33 | nM |
| Epidermal growth factor receptor erbB1 | Kd | ‘=’ | 0.38 | nM |
| Epidermal growth factor receptor erbB1 | Kd | ‘=’ | 0.41 | nM |
| Epidermal growth factor receptor erbB1 | Kd | ‘=’ | 0.42 | nM |
| Epidermal growth factor receptor erbB1 | Kd | ‘=’ | 0.44 | nM |
| Epidermal growth factor receptor erbB1 | Kd | ‘=’ | 0.44 | nM |
| Epidermal growth factor receptor erbB1 | Kd | ‘=’ | 1.00 | nM |
| Serine/threonine-protein kinase GAK | Kd | ‘=’ | 1.50 | nM |
| Mitogen-activated protein kinase kinase kinase kinase 5 | Kd | ‘=’ | 3.70 | nM |
| Serine/threonine-protein kinase GAK | Kd | ‘=’ | 6.40 | nM |
| Serine/threonine-protein kinase GAK | Kd | ‘=’ | 6.40 | nM |
| Mitogen-activated protein kinase kinase kinase kinase 5 | Kd | ‘=’ | 10.00 | nM |
| Epidermal growth factor receptor erbB1 | Kd | ‘=’ | 12.00 | nM |
| Tubulin alpha-1 chain | Kd | ‘=’ | 21.00 | nM |
| Tyrosine-protein kinase JAK3 | Kd | ‘=’ | 25.00 | nM |
| Mitogen-activated protein kinase kinase kinase kinase 5 | Kd | ‘=’ | 42.00 | nM |
| Tyrosine-protein kinase LCK | Kd | ‘=’ | 44.00 | nM |
| TRAF2- and NCK-interacting kinase | Kd | ‘=’ | 45.00 | nM |
| Serine/threonine-protein kinase 17A | Kd | ‘=’ | 57.00 | nM |
| Myotonin-protein kinase | Kd | ‘=’ | 59.00 | nM |
| Receptor protein-tyrosine kinase erbB-2 | Kd | ‘=’ | 77.00 | nM |
| Tyrosine-protein kinase BLK | Kd | ‘=’ | 78.00 | nM |
| Casein kinase I epsilon | Kd | ‘=’ | 97.00 | nM |
| Mitogen-activated protein kinase kinase kinase 1 | Kd | ‘=’ | 97.00 | nM |
| Tyrosine-protein kinase LCK | Kd | ‘=’ | 99.00 | nM |
| Casein kinase I epsilon | Kd | ‘=’ | 100.00 | nM |
| Serine/threonine-protein kinase 10 | Kd | ‘=’ | 110.00 | nM |
| Tyrosine-protein kinase ABL | Kd | ‘=’ | 110.00 | nM |
| Serine/threonine-protein kinase GAK | Kd | ‘=’ | 117.00 | nM |
| Tyrosine-protein kinase SRC | Kd | ‘=’ | 120.00 | nM |
| Mitogen-activated protein kinase kinase kinase 4 | Kd | ‘=’ | 130.00 | nM |
| Tyrosine-protein kinase ABL | Kd | ‘=’ | 130.00 | nM |
| Serine/threonine-protein kinase NEK2 | Kd | ‘=’ | 140.00 | nM |
| Tyrosine-protein kinase ABL2 | Kd | ‘=’ | 160.00 | nM |
| Tyrosine-protein kinase ABL | Kd | ‘=’ | 160.00 | nM |
| TRAF2- and NCK-interacting kinase | Kd | ‘=’ | 170.00 | nM |
| Mitogen-activated protein kinase kinase kinase kinase 3 | Kd | ‘=’ | 170.00 | nM |
| Tyrosine-protein kinase ABL | Kd | ‘=’ | 170.00 | nM |
| Serine/threonine-protein kinase WEE1 | Kd | ‘=’ | 172.00 | nM |
| Tyrosine-protein kinase ABL | Kd | ‘=’ | 180.00 | nM |
| Mitogen-activated protein kinase kinase kinase kinase 3 | Kd | ‘=’ | 189.00 | nM |
| Tyrosine-protein kinase ABL | Kd | ‘=’ | 190.00 | nM |
| Tyrosine-protein kinase FRK | Kd | ‘=’ | 190.00 | nM |
| Tyrosine-protein kinase FRK | Kd | ‘=’ | 190.00 | nM |
| Serine/threonine-protein kinase 17A | Kd | ‘=’ | 200.00 | nM |
| Tyrosine-protein kinase ABL | Kd | ‘=’ | 220.00 | nM |
| Tyrosine-protein kinase ABL | Kd | ‘=’ | 230.00 | nM |
| Tyrosine-protein kinase FER | Kd | ‘=’ | 250.00 | nM |
| Serine/threonine-protein kinase 2 | Kd | ‘=’ | 250.00 | nM |
| Mitogen-activated protein kinase kinase kinase kinase 1 | Kd | ‘=’ | 270.00 | nM |
| Mitogen-activated protein kinase kinase kinase 4 | Kd | ‘=’ | 280.00 | nM |
| Tyrosine-protein kinase SRC | Kd | ‘=’ | 280.00 | nM |
| Eukaryotic translation initiation factor 2-alpha kinase 4 | Kd | ‘=’ | 290.00 | nM |
| Tyrosine-protein kinase ABL | Kd | ‘=’ | 300.00 | nM |
| Citron Rho-interacting kinase | Kd | ‘=’ | 310.00 | nM |
| Serine/threonine-protein kinase 10 | Kd | ‘=’ | 330.00 | nM |
| Mitogen-activated protein kinase kinase kinase kinase 4 | Kd | ‘=’ | 330.00 | nM |
| Dual specificity mitogen-activated protein kinase kinase 1 | Kd | ‘=’ | 360.00 | nM |
| Serine/threonine-protein kinase 2 | Kd | ‘=’ | 360.00 | nM |
| Mitogen-activated protein kinase kinase kinase 4 | Kd | ‘=’ | 369.00 | nM |
| Tyrosine-protein kinase ABL2 | Kd | ‘=’ | 370.00 | nM |
| Ephrin type-A receptor 8 | Kd | ‘=’ | 400.00 | nM |
| Tyrosine-protein kinase ABL | Kd | ‘=’ | 430.00 | nM |
| Wee1-like protein kinase 2 | Kd | ‘=’ | 498.00 | nM |
| Receptor protein-tyrosine kinase erbB-2 | Kd | ‘=’ | 500.00 | nM |
| Tyrosine-protein kinase BTK | Kd | ‘=’ | 514.00 | nM |
| BMP-2-inducible protein kinase | Kd | ‘=’ | 540.00 | nM |
| Tyrosine-protein kinase ABL | Kd | ‘=’ | 540.00 | nM |
| Tyrosine-protein kinase ABL | Kd | ‘=’ | 560.00 | nM |
| Tyrosine-protein kinase FRK | Kd | ‘=’ | 680.00 | nM |
| Serine/threonine-protein kinase NEK2 | Kd | ‘=’ | 680.00 | nM |
| Dual specificity protein kinase CLK2 | Kd | ‘=’ | 700.00 | nM |
| Ephrin type-A receptor 5 | Kd | ‘=’ | 710.00 | nM |
| Tyrosine-protein kinase Lyn | Kd | ‘=’ | 720.00 | nM |
| Serine/threonine-protein kinase WEE1 | Kd | ‘=’ | 770.00 | nM |
| Dual specificity mitogen-activated protein kinase kinase 2 | Kd | ‘=’ | 810.00 | nM |
| Tyrosine-protein kinase YES | Kd | ‘=’ | 840.00 | nM |
| Tyrosine-protein kinase ABL | Kd | ‘=’ | 860.00 | nM |
| Mixed lineage kinase 7 | Kd | ‘=’ | 875.00 | nM |
| Tyrosine kinase non-receptor protein 2 | Kd | ‘=’ | 890.00 | nM |
| Tyrosine-protein kinase receptor UFO | Kd | ‘=’ | 920.00 | nM |
| Tyrosine- and threonine-specific cdc2-inhibitory kinase | Kd | ‘=’ | 930.00 | nM |
| Tyrosine-protein kinase FGR | Kd | ‘=’ | 950.00 | nM |
| Tyrosine-protein kinase Lyn | Kd | ‘=’ | 960.00 | nM |
| Serine/threonine-protein kinase WEE1 | Kd | ‘=’ | 967.00 | nM |
| Tyrosine-protein kinase receptor Tie-1 | Kd | ‘=’ | 1000.00 | nM |
| Casein kinase I isoform alpha-like | Kd | ‘=’ | 1000.00 | nM |
| Dual specificty protein kinase CLK1 | Kd | ‘=’ | 1000.00 | nM |

The present disclosure also provide a companion diagnostic composition for determining the sensitivity of an EGFR inhibitor drug, the companion diagnostic composition comprising an agent for measuring an RNA expression level of a LEPRE1 gene or an agent for specifying a protein expression level of the LEPRE1 gene, wherein,
the agent for measuring an RNA expression level of a LEPRE1 gene is preferably selected from the group consisting of a sense primer, an antisense primer and a probe that complementarily bind to the LEPRE1 gene or RNA thereof;
the agent for specifying a protein expression level of the LEPRE1 gene is preferably selected from the group consisting of an antibody, an aptamer and a probe that specifically binds to a protein encoded by the LEPRE1 gene; and
the companion diagnostic composition comprises at least one alteration selected from Table 1 that predicts the expression level of the LEPRE1 gene.

TABLE 1

| Chromosome | Location | Reference (Ref.) | Alteration (Alt.) | Type of alteration |
|---|---|---|---|---|
| 1 | 205849911 | C | T | Substitution |
| 1 | 205849976 | G | A | Substitution |
| 4 | 68337362 | T | C | Substitution |
| 4 | 182680440 | G | C | Substitution |
| 4 | 182680513 | G | A | Substitution |
| 20 | 32983679 | C | T | Substitution |

In a further embodiment, the present disclosure provides a method of discovering a gene for determining drug sensitivity, the method comprising:
(A) selecting a candidate gene for determining responsiveness to a target drug through cancer companion diagnostic marker scanning (GBLscan);
(B) realizing an overexpressed state of the candidate gene in a cell line targeted by the target drug;
(C) calculating responsiveness of the target drug to the targeted cell line, in the state in which the candidate gene is overexpressed, as obtained in (B);
(D) realizing an under expressed state of the candidate gene in the cell line targeted by the target drug;
(E) calculating responsiveness of the target drug to the targeted cell line, in the state in which the candidate gene is under expressed, obtained in (D); and
(F) verifying whether or not the candidate gene is a marker for determining responsiveness to the target drug, compared with the responsiveness calculated in (C) and (E), wherein,
in (B), the overexpressed state of the candidate gene is preferably realized using pcDNA3.1;
in (D), the underexpressed state of the candidate gene is preferably realized using siRNA;
in (C) and (E), the responsiveness is preferably calculated through IC50 values of the target drug for the targeted cell line;
the target drug is preferably an EGFR inhibitor drug; and
the candidate gene is preferably a LEPRE1 gene.

The present disclosure has experimentally confirmed a biomarker predicted through a system (GBLscan) for predicting the correlation between a cancer drug and the expression information and gene copy number variation of a cell line genome, through collection information-based linear regression modeling of quantitative trait loci and deep machine learning, and is to provide reliability by verifying prediction through a machine learning system. The expression level of the LEPRE1 gene in cancer cells with high EGFR expression shows the example of a verification experiment for determining sensitivity to a tyrosine kinase inhibitor anticancer agent.

The present disclosure also provides a model for verifying a tyrosine kinase inhibitor biomarker, thus enabling other drugs and other genes to be verified in the same manner in the future.

According to the characteristics of the present disclosure for achieving the above-described objects, the present disclosure largely comprises two steps of: predicting the expression information and gene copy number variation of a cell line genome and drug responsiveness; and verifying whether an alteration predicted through a system actually affects drug sensitivity. In the prediction step, GBLscan, which is a program (a machine learning system) developed by the present applicant, was used, and experimental verification was carried out through joint research with the Safety Evaluation Institute, which is a national verification organization.

As described above, according to the present disclosure, from the in-vivo or in-vitro sensitivity results of drugs of which the genetic information is known, it was experimentally verified whether the alteration or copy number variation and a change in the expression level of a resultant predicted by GBLscan, which has the effect of predicting the degree of sensitivity to drugs of which the pharmacological effects are unidentified, actually affect drug responsiveness.

### EXAMPLES

According to exemplary embodiments of the present disclosure, the drug sensitivity in a cancer cell line is determined according to the expression level of a gene, wherein the gene is a LEPRE1 gene, and the drug is an EGFR inhibitor drug, drug sensitivity is determined such that the sensitivity of an EGFR inhibitor drug in the cancer cell line is high according to the overexpression level of the LEPRE1 gene, and the resistance of an EGFR inhibitor drug to the cancer cell line is high according to the under expression level of the LEPRE1 gene, wherein the EGFR inhibitor drug is pelitinib, and the cancer cell line is any at least one selected from THP-1 and KG-1, which are hematological cancer cell lines, and A549, which is a lung cancer cell line, and the expression level of the LEPRE1 gene is determined by detecting at least one alteration from the Table 1.

TABLE 1

| Chromosome | Location | Reference (Ref.) | Alteration (Alt.) | Type of alteration |
|---|---|---|---|---|
| 1 | 205849911 | C | T | Substitution |
| 1 | 205849976 | G | A | Substitution |
| 4 | 68337362 | T | C | Substitution |
| 4 | 182680440 | G | C | Substitution |
| 4 | 182680513 | G | A | Substitution |
| 20 | 32983679 | C | T | Substitution |

Accordingly, in the present disclosure, from the in-vivo or in-vitro sensitivity results of drugs of which the genetic information is known, it was experimentally confirmed whether the alteration or copy number variation and a change in the expression level of a resultant predicted by GBLscan, which has the effect of predicting the degree of sensitivity to drugs of which the pharmacological effects are unidentified, actually affect drug responsiveness.

According to exemplary embodiments of the present disclosure, wherein the gene associated to drug sensitivity further comprise at least one gene selected from Table 2 below that regulate the extracellular matrix environment, such as LEPRE1. The sensitivity or resistance to the drug varies according to the expression level of the genes.

TABLE 2

| No | Gene name | Relation | No | Gene name | Relation | No | Gene name | Relation |
|---|---|---|---|---|---|---|---|---|
| 1 | A2M | Highly related | 101 | LAMB1 | Highly related | 201 | ITGA10 | Related |
| 2 | ACAN | Highly related | 102 | LAMB3 | Highly related | 202 | ITGA11 | Related |
| 3 | ADAM 10 | Highly related | 103 | LAMC1 | Highly related | 203 | ITGA2 | Related |
| 4 | ADAM 15 | Highly related | 104 | LAMC2 | Highly related | 204 | ITGA2B | Related |
| 5 | ADAM 17 | Highly related | 105 | MMP1 | Highly related | 205 | ITGA3 | Related |
| 6 | ADAM8 | Highly related | 106 | MMP10 | Highly related | 206 | ITGA4 | Related |
| 7 | ADAM9 | Highly related | 107 | MMP11 | Highly related | 207 | ITGA5 | Related |
| 8 | ADAMTS1 | Highly related | 108 | MMP12 | Highly related | 208 | ITGA6 | Related |
| 9 | ADAMTS16 | Highly related | 109 | MMP13 | Highly related | 209 | ITGA7 | Related |
| 10 | ADAMTS18 | Highly related | 110 | MMP14 | Highly related | 210 | ITGA8 | Related |
| 11 | ADAMTS4 | Highly related | 111 | MMP15 | Highly related | 211 | ITGA9 | Related |
| 12 | ADAMTS5 | Highly related | 112 | MMP16 | Highly related | 212 | ITGAD | Related |
| 13 | ADAMTS8 | Highly related | 113 | MMP17 | Highly related | 213 | ITGAE | Related |
| 14 | ADAMTS9 | Highly related | 114 | MMP19 | Highly related | 214 | ITGAL | Related |
| 15 | BCAN | Highly related | 115 | MMP2 | Highly related | 215 | ITGAM | Related |
| 16 | BMP1 | Highly related | 116 | MMP20 | Highly related | 216 | ITGAV | Related |
| 17 | BSG | Highly related | 117 | MMP24 | Highly related | 217 | ITGAX | Related |
| 18 | CAPN1 | Highly related | 118 | MMP25 | Highly related | 218 | ITGB1 | Related |
| 19 | CAPN10 | Highly related | 119 | MMP3 | Highly related | 219 | ITGB2 | Related |
| 20 | CAPN11 | Highly related | 120 | MMP7 | Highly related | 220 | ITGB3 | Related |
| 21 | CAPN12 | Highly | 121 | MMP8 | Highly | 221 | ITGB4 | Related |
| | | related | | | related | | | |
| 22 | CAPN13 | Highly related | 122 | MMP9 | Highly related | 222 | ITGB5 | Related |
| 23 | CAPN14 | Highly related | 123 | NCSTN | Highly related | 223 | ITGB6 | Related |
| 24 | CAPN15 | Highly related | 124 | NID1 | Highly related | 224 | ITGB7 | Related |
| 25 | CAPN2 | Highly related | 125 | OPTC | Highly related | 225 | ITGB8 | Related |
| 26 | CAPN3 | Highly related | 126 | PHYKPL | Highly related | 226 | JAM2 | Related |
| 27 | CAPN5 | Highly related | 127 | PLG | Highly related | 227 | JAM3 | Related |
| 28 | CAPN6 | Highly related | 128 | PRSS1 | Highly related | 228 | KDR | Related |
| 29 | CAPN7 | Highly related | 129 | PRSS2 | Highly related | 229 | LAMA1 | Related |
| 30 | CAPN8 | Highly related | 130 | PSEN1 | Highly related | 230 | LAMA2 | Related |
| 31 | CAPN9 | Highly related | 131 | SCUBE1 | Highly related | 231 | LAMA4 | Related |
| 32 | CAPNS1 | Highly related | 132 | SCUBE3 | Highly related | 232 | LAM B2 | Related |
| 33 | CAPNS2 | Highly related | 133 | SPOCK3 | Highly related | 233 | LAMC3 | Related |
| 34 | CASP3 | Highly related | 134 | SPP1 | Highly related | 234 | LOX | Related |
| 35 | CAST | Highly related | 135 | TIMP1 | Highly related | 235 | LOXL1 | Related |
| 36 | CD44 | Highly related | 136 | TIMP2 | Highly related | 236 | LOXL2 | Related |
| 37 | CDH1 | Highly related | 137 | TLL1 | Highly related | 237 | LOXL3 | Related |
| 38 | CMA1 | Highly related | 138 | TLL2 | Highly related | 238 | LOXL4 | Related |
| 39 | COL10A1 | Highly related | 139 | TMPRSS6 | Highly related | 239 | LRP4 | Related |
| 40 | COL11A1 | Highly related | 140 | TPSAB1 | Highly related | 240 | LTBP1 | Related |
| 41 | COL11A2 | Highly related | 141 | ACTN1 | Related | 241 | LTBP2 | Related |
| 42 | COL12A1 | Highly related | 142 | ADAM12 | Related | 242 | LTBP3 | Related |
| 43 | COL13A1 | Highly related | 143 | ADAM19 | Related | 243 | LTBP4 | Related |
| 44 | COL14A1 | Highly related | 144 | ADAMTS14 | Related | 244 | LUM | Related |
| 45 | COL15A1 | Highly related | 145 | ADAMTS2 | Related | 245 | MADCAM1 | Related |
| 46 | COL16A1 | Highly related | 146 | ADAMTS3 | Related | 246 | MATN1 | Related |
| 47 | COL17A1 | Highly related | 147 | AGRN | Related | 247 | MATN3 | Related |
| 48 | COL18A1 | Highly related | 148 | APP | Related | 248 | MATN4 | Related |
| 49 | COL19A1 | Highly related | 149 | ASPN | Related | 249 | MFAP2 | Related |
| 50 | COL1A1 | Highly related | 150 | BGN | Related | 250 | MFAP3 | Related |
| 51 | COL1A2 | Highly related | 151 | BMP10 | Related | 251 | MFAP4 | Related |
| 52 | COL23A1 | Highly related | 152 | BMP2 | Related | 252 | MFAP5 | Related |
| 53 | COL25A1 | Highly related | 153 | BMP4 | Related | 253 | MUSK | Related |
| 54 | COL26A1 | Highly | 154 | BMP7 | Related | 254 | NCAM1 | Related |
| | | related | | | | | | |
| 55 | COL2A1 | Highly related | 155 | CASK | Related | 255 | NCAN | Related |
| 56 | COL3A1 | Highly related | 156 | CD151 | Related | 256 | NID2 | Related |
| 57 | COL4A1 | Highly related | 157 | CD47 | Related | 257 | NRXN1 | Related |
| 58 | COL4A2 | Highly related | 158 | CEACAM1 | Related | 258 | NTN4 | Related |
| 59 | COL4A3 | Highly related | 159 | CEACAM6 | Related | 259 | P3H1 | Related |
| 60 | COL4A4 | Highly related | 160 | CEACAM8 | Related | 260 | P3H2 | Related |
| 61 | COL4A5 | Highly related | 161 | COL20A1 | Related | 261 | P3H3 | Related |
| 62 | COL4A6 | Highly related | 162 | COL21A1 | Related | 262 | P4HA1 | Related |
| 63 | COL5A1 | Highly related | 163 | COL22A1 | Related | 263 | P4HA2 | Related |
| 64 | COL5A2 | Highly related | 164 | COL24A1 | Related | 264 | P4HA3 | Related |
| 65 | COL5A3 | Highly related | 165 | COL27A1 | Related | 265 | P4HB | Related |
| 66 | COL6A1 | Highly related | 166 | COL28A1 | Related | 266 | PCOLCE | Related |
| 67 | COL6A2 | Highly related | 167 | COLGALT1 | Related | 267 | PCOLCE2 | Related |
| 68 | COL6A3 | Highly related | 168 | COLGALT2 | Related | 268 | PDGFA | Related |
| 69 | COL6A5 | Highly related | 169 | COMP | Related | 269 | PDGFB | Related |
| 70 | COL6A6 | Highly related | 170 | CRTAP | Related | 270 | PECAM1 | Related |
| 71 | COL7A1 | Highly related | 171 | DAG1 | Related | 271 | PLEC | Related |
| 72 | COL8A1 | Highly related | 172 | DDR1 | Related | 272 | PLOD1 | Related |
| 73 | COL8A2 | Highly related | 173 | DDR2 | Related | 273 | PLOD2 | Related |
| 74 | COL9A1 | Highly related | 174 | DMD | Related | 274 | PLOD3 | Related |
| 75 | COL9A2 | Highly related | 175 | DMP1 | Related | 275 | PPIB | Related |
| 76 | COL9A3 | Highly related | 176 | DSPP | Related | 276 | PRKCA | Related |
| 77 | CTRB1 | Highly related | 177 | DST | Related | 277 | PTPRS | Related |
| 78 | CTRB2 | Highly related | 178 | EFEMP1 | Related | 278 | PXDN | Related |
| 79 | CTSB | Highly related | 179 | EFEMP2 | Related | 279 | SDC1 | Related |
| 80 | CTSD | Highly related | 180 | EMILIN1 | Related | 280 | SDC2 | Related |
| 81 | CTSG | Highly related | 181 | EMILIN2 | Related | 281 | SDC3 | Related |
| 82 | CTSK | Highly related | 182 | EMILIN3 | Related | 282 | SDC4 | Related |
| 83 | CTSL | Highly related | 183 | F11R | Related | 283 | SERPINE1 | Related |
| 84 | CTSS | Highly related | 184 | FBLN1 | Related | 284 | SERPINH1 | Related |
| 85 | CTSV | Highly related | 185 | FBLN2 | Related | 285 | SH3PXD2A | Related |
| 86 | DCN | Highly related | 186 | FBLN5 | Related | 286 | SPARC | Related |
| 87 | ELANE | Highly | 187 | FGA | Related | 287 | TGFB1 | Related |
| | | related | | | | | | |
| 88 | ELN | Highly related | 188 | FGB | Related | 288 | TGFB2 | Related |
| 89 | FBN1 | Highly related | 189 | FGF2 | Related | 289 | TGFB3 | Related |
| 90 | FBN2 | Highly related | 190 | FGG | Related | 290 | THBS1 | Related |
| 91 | FBN3 | Highly related | 191 | FMOD | Related | 291 | TNC | Related |
| 92 | FN1 | Highly related | 192 | GDF5 | Related | 292 | TNN | Related |
| 93 | FURIN | Highly related | 193 | HAPLN1 | Related | 293 | TNR | Related |
| 94 | HSPG2 | Highly related | 194 | IBSP | Related | 294 | TNXB | Related |
| 95 | HTRA1 | Highly related | 195 | ICAM1 | Related | 295 | TRAPPC4 | Related |
| 96 | KLK2 | Highly related | 196 | ICAM2 | Related | 296 | TTR | Related |
| 97 | KLK7 | Highly related | 197 | ICAM3 | Related | 297 | VCAM1 | Related |
| 98 | KLKB1 | Highly related | 198 | ICAM4 | Related | 298 | VCAN | Related |
| 99 | LAMA3 | Highly related | 199 | ICAM5 | Related | 299 | VTN | Related |
| 100 | LAMA5 | Highly related | 200 | ITGA1 | Related | 300 | VWF | Related |

According to exemplary embodiments of the present disclosure, wherein the gene associated to drug sensitivity is selected from the genes selected from Table 3 below that regulate the metabolism of collagen, such as LEPRE1, and determining drug sensitivity such that the sensitivity of the EGFR inhibitor drug to the cancer cell line changes according to the expression level of the genes. The sensitivity or resistance to the drug varies according to the expression level of the genes.

TABLE 3

| No | Gene name | Relation | No | Gene name | Relation | No | Gene namel | Relation |
|---|---|---|---|---|---|---|---|---|
| 1 | BMP1 | Highly related | 49 | ADAM 17 | Related | 97 | KLK8 | Related |
| 2 | COL10A1 | Highly related | 50 | ADAM9 | Related | 98 | KLK9 | Related |
| 3 | COL11A1 | Highly related | 51 | ADAMTS14 | Related | 99 | KLKB1 | Related |
| 4 | COL11A2 | Highly related | 52 | ADAMTS2 | Related | 100 | KNG1 | Related |
| 5 | COL12A1 | Highly related | 53 | ADAMTS3 | Related | 101 | LAMA3 | Related |
| 6 | COL13A1 | Highly related | 54 | CD151 | Related | 102 | LAM B3 | Related |
| 7 | COL14A1 | Highly related | 55 | COL28A1 | Related | 103 | LAMC2 | Related |
| 8 | COL15A1 | Highly related | 56 | COLGALT1 | Related | 104 | LOX | Related |
| 9 | COL16A1 | Highly related | 57 | COLGALT2 | Related | 105 | LOXL1 | Related |
| 10 | COL17A1 | Highly related | 58 | CRTAP | Related | 106 | LOXL2 | Related |
| 11 | COL18A1 | Highly related | 59 | CTSB | Related | 107 | LOXL3 | Related |
| 12 | COL19A1 | Highly related | 60 | CTSD | Related | 108 | LOXL4 | Related |
| 13 | COL1A1 | Highly related | 61 | CTSK | Related | 109 | MMP1 | Related |
| 14 | COL1A2 | Highly related | 62 | CTSL | Related | 110 | MMP10 | Related |
| 15 | COL20A1 | Highly related | 63 | CTSS | Related | 111 | MMP11 | Related |
| 16 | COL21A1 | Highly related | 64 | CTSV | Related | 112 | MMP12 | Related |
| 17 | COL22A1 | Highly related | 65 | DST | Related | 113 | MMP13 | Related |
| 18 | COL23A1 | Highly related | 66 | ELANE | Related | 114 | MMP14 | Related |
| 19 | COL24A1 | Highly related | 67 | F10 | Related | 115 | MMP15 | Related |
| 20 | COL25A1 | Highly related | 68 | F11 | Related | 116 | MMP19 | Related |
| 21 | COL26A1 | Highly related | 69 | F12 | Related | 117 | MMP2 | Related |
| 22 | COL27A1 | Highly related | 70 | F13A1 | Related | 118 | MMP20 | Related |
| 23 | COL2A1 | Highly related | 71 | F13B | Related | 119 | MMP3 | Related |
| 24 | COL3A1 | Highly related | 72 | F2 | Related | 120 | MMP7 | Related |
| 25 | COL4A1 | Highly related | 73 | F3 | Related | 121 | MMP8 | Related |
| 26 | COL4A2 | Highly related | 74 | F5 | Related | 122 | MMP9 | Related |
| 27 | COL4A3 | Highly related | 75 | F7 | Related | 123 | P3H1 | Related |
| 28 | COL4A4 | Highly related | 76 | F8 | Related | 124 | P3H2 | Related |
| 29 | COL4A5 | Highly related | 77 | F9 | Related | 125 | P3H3 | Related |
| 30 | COL4A6 | Highly related | 78 | FGA | Related | 126 | P4HA1 | Related |
| 31 | COL5A1 | Highly | 79 | FGB | Related | 127 | P4HA2 | Related |
| | | related | | | | | | |
| 32 | COL5A2 | Highly related | 80 | FGG | Related | 128 | P4HA3 | Related |
| 33 | COL5A3 | Highly related | 81 | FURIN | Related | 129 | P4HB | Related |
| 34 | COL6A1 | Highly related | 82 | ITGA6 | Related | 130 | PCOLCE2 | Related |
| 35 | COL6A2 | Highly related | 83 | ITGB4 | Related | 131 | PHYKPL | Related |
| 36 | COL6A3 | Highly related | 84 | KLK1 | Related | 132 | PLEC | Related |
| 37 | COL6A5 | Highly related | 85 | KLK10 | Related | 133 | PLOD1 | Related |
| 38 | COL6A6 | Highly related | 86 | KLK11 | Related | 134 | PLOD2 | Related |
| 39 | COL7A1 | Highly related | 87 | KLK12 | Related | 135 | PLOD3 | Related |
| 40 | COL8A1 | Highly related | 88 | KLK13 | Related | 136 | PPIB | Related |
| 41 | COL8A2 | Highly related | 89 | KLK14 | Related | 137 | PROC | Related |
| 42 | COL9A1 | Highly related | 90 | KLK15 | Related | 138 | PROS1 | Related |
| 43 | COL9A2 | Highly related | 91 | KLK2 | Related | 139 | PRSS2 | Related |
| 44 | COL9A3 | Highly related | 92 | KLK3 | Related | 140 | PXDN | Related |
| 45 | PCOLCE | Highly related | 93 | KLK4 | Related | 141 | SERPINC1 | Related |
| 46 | TLL1 | Highly related | 94 | KLK5 | Related | 142 | SERPINH1 | Related |
| 47 | TLL2 | Highly related | 95 | KLK6 | Related | 143 | TFPI | Related |
| 48 | ADAM 10 | Related | 96 | KLK7 | Related | 144 | THBD | Related |
| | | | | | | 145 | TMPRSS6 | Related |

Another exemplary embodiments of the present disclosure provides a method to determine a sensitivity or resistance to a drug comprising:
measuring an expression level of a gene in a cancer cell line; and
determine the sensitivity is determined high based on overexpression of the gene and the resistance is determined high based on underexpression of the gene, wherein
the gene is LEPRE1 gene or one selected from Table 1, Table 2, or Table 3;
the drug is an EGFR inhibitor or a drug having multi-target efficacies as shown in Table 4 or an efficacy profile similar thereto, with 60% or higher, preferably 70% or higher, more preferably 85%, most preferably 90% or higher similarity. The sensitivity or resistance to the drug varies according to the expression level of the genes.

TABLE 4

| Target Name | Standard Type | Standard Relation | Standard Value | Standard Units |
|---|---|---|---|---|
| Epidermal growth factor receptor erbB1 | Kd | ‘=’ | 0.23 | nM |
| Epidermal growth factor receptor erbB1 | Kd | ‘=’ | 0.24 | nM |
| Epidermal growth factor receptor erbB1 | Kd | ‘=’ | 0.24 | nM |
| Epidermal growth factor receptor erbB1 | Kd | ‘=’ | 0.27 | nM |
| Epidermal growth factor receptor erbB1 | Kd | ‘=’ | 0.33 | nM |
| Epidermal growth factor receptor erbB1 | Kd | ‘=’ | 0.38 | nM |
| Epidermal growth factor receptor erbB1 | Kd | ‘=’ | 0.41 | nM |
| Epidermal growth factor receptor erbB1 | Kd | ‘=’ | 0.42 | nM |
| Epidermal growth factor receptor erbB1 | Kd | ‘=’ | 0.44 | nM |
| Epidermal growth factor receptor erbB1 | Kd | ‘=’ | 0.44 | nM |
| Epidermal growth factor receptor erbB1 | Kd | ‘=’ | 1.00 | nM |
| Serine/threonine-protein kinase GAK | Kd | ‘=’ | 1.50 | nM |
| Mitogen-activated protein kinase kinase kinase kinase 5 | Kd | ‘=’ | 3.70 | nM |
| Serine/threonine-protein kinase GAK | Kd | ‘=’ | 6.40 | nM |
| Serine/threonine-protein kinase GAK | Kd | ‘=’ | 6.40 | nM |
| Mitogen-activated protein kinase kinase kinase kinase 5 | Kd | ‘=’ | 10.00 | nM |
| Epidermal growth factor receptor erbB1 | Kd | ‘=’ | 12.00 | nM |
| Tubulin alpha-1 chain | Kd | ‘=’ | 21.00 | nM |
| Tyrosine-protein kinase JAK3 | Kd | ‘=’ | 25.00 | nM |
| Mitogen-activated protein kinase kinase kinase kinase 5 | Kd | ‘=’ | 42.00 | nM |
| Tyrosine-protein kinase LCK | Kd | ‘=’ | 44.00 | nM |
| TRAF2- and NCK-interacting kinase | Kd | ‘=’ | 45.00 | nM |
| Serine/threonine-protein kinase 17A | Kd | ‘=’ | 57.00 | nM |
| Myotonin-protein kinase | Kd | ‘=’ | 59.00 | nM |
| Receptor protein-tyrosine kinase erbB-2 | Kd | ‘=’ | 77.00 | nM |
| Tyrosine-protein kinase BLK | Kd | ‘=’ | 78.00 | nM |
| Casein kinase I epsilon | Kd | ‘=’ | 97.00 | nM |
| Mitogen-activated protein kinase kinase kinase 1 | Kd | ‘=’ | 97.00 | nM |
| Tyrosine-protein kinase LCK | Kd | ‘=’ | 99.00 | nM |
| Casein kinase I epsilon | Kd | ‘=’ | 100.00 | nM |
| Serine/threonine-protein kinase 10 | Kd | ‘=’ | 110.00 | nM |
| Tyrosine-protein kinase ABL | Kd | ‘=’ | 110.00 | nM |
| Serine/threonine-protein kinase GAK | Kd | ‘=’ | 117.00 | nM |
| Tyrosine-protein kinase SRC | Kd | ‘=’ | 120.00 | nM |
| Mitogen-activated protein kinase kinase kinase 4 | Kd | ‘=’ | 130.00 | nM |
| Tyrosine-protein kinase ABL | Kd | ‘=’ | 130.00 | nM |
| Serine/threonine-protein kinase NEK2 | Kd | ‘=’ | 140.00 | nM |
| Tyrosine-protein kinase ABL2 | Kd | ‘=’ | 160.00 | nM |
| Tyrosine-protein kinase ABL | Kd | ‘=’ | 160.00 | nM |
| TRAF2- and NCK-interacting kinase | Kd | ‘=’ | 170.00 | nM |
| Mitogen-activated protein kinase kinase kinase kinase 3 | Kd | ‘=’ | 170.00 | nM |
| Tyrosine-protein kinase ABL | Kd | ‘=’ | 170.00 | nM |
| Serine/threonine-protein kinase WEE1 | Kd | ‘=’ | 172.00 | nM |
| Tyrosine-protein kinase ABL | Kd | ‘=’ | 180.00 | nM |
| Mitogen-activated protein kinase kinase kinase kinase 3 | Kd | ‘=’ | 189.00 | nM |
| Tyrosine-protein kinase ABL | Kd | ‘=’ | 190.00 | nM |
| Tyrosine-protein kinase FRK | Kd | ‘=’ | 190.00 | nM |
| Tyrosine-protein kinase FRK | Kd | ‘=’ | 190.00 | nM |
| Serine/threonine-protein kinase 17A | Kd | ‘=’ | 200.00 | nM |
| Tyrosine-protein kinase ABL | Kd | ‘=’ | 220.00 | nM |
| Tyrosine-protein kinase ABL | Kd | ‘=’ | 230.00 | nM |
| Tyrosine-protein kinase FER | Kd | ‘=’ | 250.00 | nM |
| Serine/threonine-protein kinase 2 | Kd | ‘=’ | 250.00 | nM |
| Mitogen-activated protein kinase kinase kinase kinase 1 | Kd | ‘=’ | 270.00 | nM |
| Mitogen-activated protein kinase kinase kinase 4 | Kd | ‘=’ | 280.00 | nM |
| Tyrosine-protein kinase SRC | Kd | ‘=’ | 280.00 | nM |
| Eukaryotic translation initiation factor 2-alpha kinase 4 | Kd | ‘=’ | 290.00 | nM |
| Tyrosine-protein kinase ABL | Kd | ‘=’ | 300.00 | nM |
| Citron Rho-interacting kinase | Kd | ‘=’ | 310.00 | nM |
| Serine/threonine-protein kinase 10 | Kd | ‘=’ | 330.00 | nM |
| Mitogen-activated protein kinase kinase kinase kinase 4 | Kd | ‘=’ | 330.00 | nM |
| Dual specificity mitogen-activated protein kinase kinase 1 | Kd | ‘=’ | 360.00 | nM |
| Serine/threonine-protein kinase 2 | Kd | ‘=’ | 360.00 | nM |
| Mitogen-activated protein kinase kinase kinase 4 | Kd | ‘=’ | 369.00 | nM |
| Tyrosine-protein kinase ABL2 | Kd | ‘=’ | 370.00 | nM |
| Ephrin type-A receptor 8 | Kd | ‘=’ | 400.00 | nM |
| Tyrosine-protein kinase ABL | Kd | ‘=’ | 430.00 | nM |
| Wee1-like protein kinase 2 | Kd | ‘=’ | 498.00 | nM |
| Receptor protein-tyrosine kinase erbB-2 | Kd | ‘=’ | 500.00 | nM |
| Tyrosine-protein kinase BTK | Kd | ‘=’ | 514.00 | nM |
| BMP-2-inducible protein kinase | Kd | ‘=’ | 540.00 | nM |
| Tyrosine-protein kinase ABL | Kd | ‘=’ | 540.00 | nM |
| Tyrosine-protein kinase ABL | Kd | ‘=’ | 560.00 | nM |
| Tyrosine-protein kinase FRK | Kd | ‘=’ | 680.00 | nM |
| Serine/threonine-protein kinase NEK2 | Kd | ‘=’ | 680.00 | nM |
| Dual specificity protein kinase CLK2 | Kd | ‘=’ | 700.00 | nM |
| Ephrin type-A receptor 5 | Kd | ‘=’ | 710.00 | nM |
| Tyrosine-protein kinase Lyn | Kd | ‘=’ | 720.00 | nM |
| Serine/threonine-protein kinase WEE1 | Kd | ‘=’ | 770.00 | nM |
| Dual specificity mitogen-activated protein kinase kinase 2 | Kd | ‘=’ | 810.00 | nM |
| Tyrosine-protein kinase YES | Kd | ‘=’ | 840.00 | nM |
| Tyrosine-protein kinase ABL | Kd | ‘=’ | 860.00 | nM |
| Mixed lineage kinase 7 | Kd | ‘=’ | 875.00 | nM |
| Tyrosine kinase non-receptor protein 2 | Kd | ‘=’ | 890.00 | nM |
| Tyrosine-protein kinase receptor UFO | Kd | ‘=’ | 920.00 | nM |
| Tyrosine- and threonine-specific cdc2-inhibitory kinase | Kd | ‘=’ | 930.00 | nM |
| Tyrosine-protein kinase FGR | Kd | ‘=’ | 950.00 | nM |
| Tyrosine-protein kinase Lyn | Kd | ‘=’ | 960.00 | nM |
| Serine/threonine-protein kinase WEE1 | Kd | ‘=’ | 967.00 | nM |
| Tyrosine-protein kinase receptor Tie-1 | Kd | ‘=’ | 1000.00 | nM |
| Casein kinase I isoform alpha-like | Kd | ‘=’ | 1000.00 | nM |
| Dual specificty protein kinase CLK1 | Kd | ‘=’ | 1000.00 | nM |

In the present disclosure, examples provides if the expression of the LEPRE1 gene predicted through a drug response prediction system using an artificial intelligence deep learning model based on combined data of cell line alterations and cancer drugs, according to the present disclosure, actually affects cancer drug responsiveness.

Epidermal growth factor receptor is a member of the ErbB receptor family and consists of four closely related receptors (the subfamily of tyrosine kinases: EGFR (ErbB-1); HER2/neu (ErbB-2); Her 3 (ErbB-3); and Her 4 (ErbB-4). In addition, in many cancer types, mutations that affect EGFR expression or activity can cause cancer.

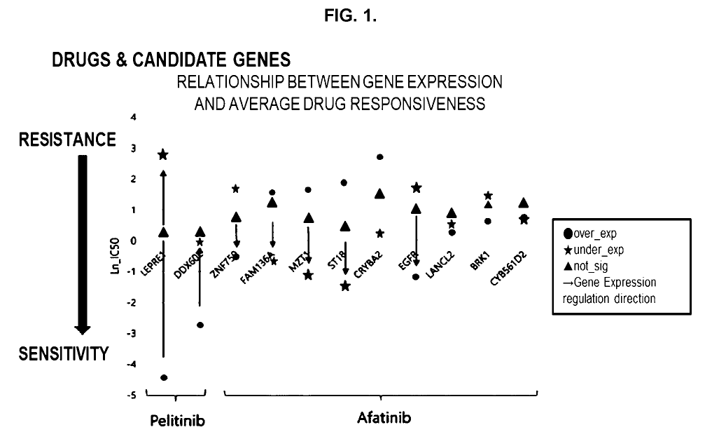

As illustrated in FIG. 1, genes, of which the overexpression and inhibited expression may affect drug resistance and sensitivity, and drugs were selected using the core technology of GBLscan as described above. It was predicted that the overexpression of the LEPRE1 gene increases drug responsiveness to peletinib, which is an EGFR TK inhibitor, whereas the inhibited expression of the LEPRE1 gene induces resistance to peletinib. LEPRE1, which is a gene whose overexpression and inhibited expression can remarkably affect drug responsiveness, was selected in this step, and was verified through the following experiments.

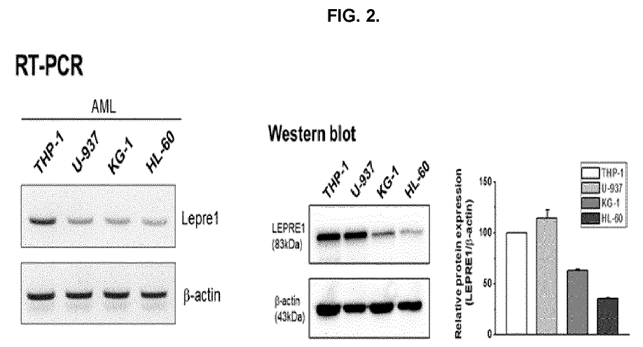

As illustrated in FIG. 2, to find a LEPRE1-underexpressing hematological cancer cell line in hematological cancer as a target carcinoma, KG-1, U-937, and HL-60, which are acute myeloid leukemia (AML) cell lines, were selected as candidate cell lines. As a result of RT-PCR, the LEPRE1 gene was most highly expressed in the THP-1 cell line. It was confirmed that, at the protein level, LEPRE1 showed the highest expression level in U-937 and was under-expressed in HL-60.

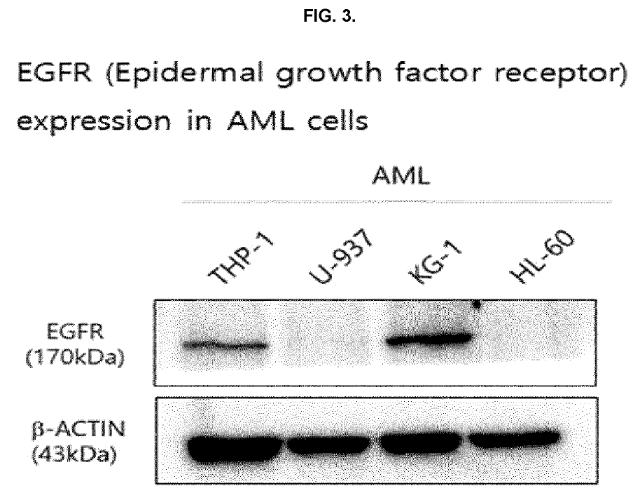

As illustrated in FIG. 3, since pelitinib, which is a drug to be used in experiments, is an EGFR tyrosine kinase-targeting drug, the expression of EGFR was confirmed in AML cell lines. As a result, the expression of EGFR in KG-1 and THP-1 was confirmed. Thus, THP-1 was selected as a LEPRE1-overexpressed cell line, and KG-1 was selected as a LEPRE1-underexpressed cell line, and the subsequent processes were carried out.

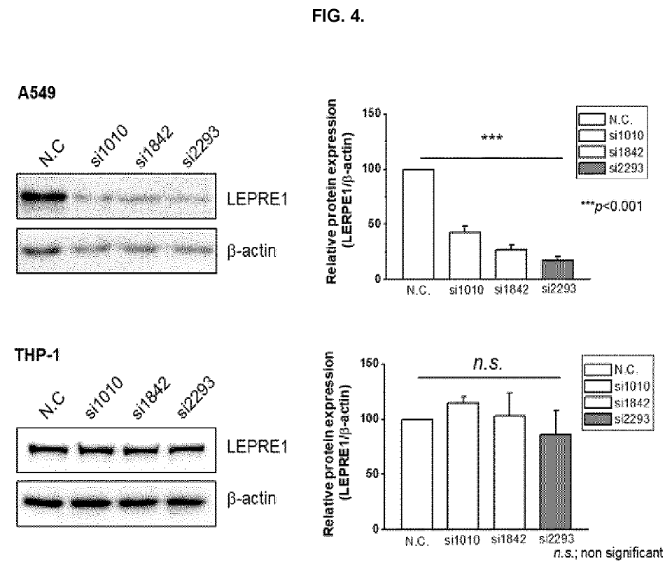

As illustrated in FIG. 4, experiments for the inhibited expression of the LEPRE1 gene through siRNA in a lung cancer cell line (A549) and a hematological cancer cell line (THP-1) were carried out. The expression level of LEPRE1 in A549 decreased by 83% when si2293 siRNA was used, whereas the expression level of LEPRE1 in THP-1 decreased by 15% when si2293 siRNA was used. In the case of the A549 cell line, which is an adherent cell, it was easy to create conditions for inhibited expression through siRNA, whereas the THP-1 cell line, which is a floating cell, had poorer efficiency in carrying out the inhibition of expression through siRNA than the A549 cell line. Accordingly, it was decided to first verify a change in drug responsiveness of the A549 cell line according to the expression level.

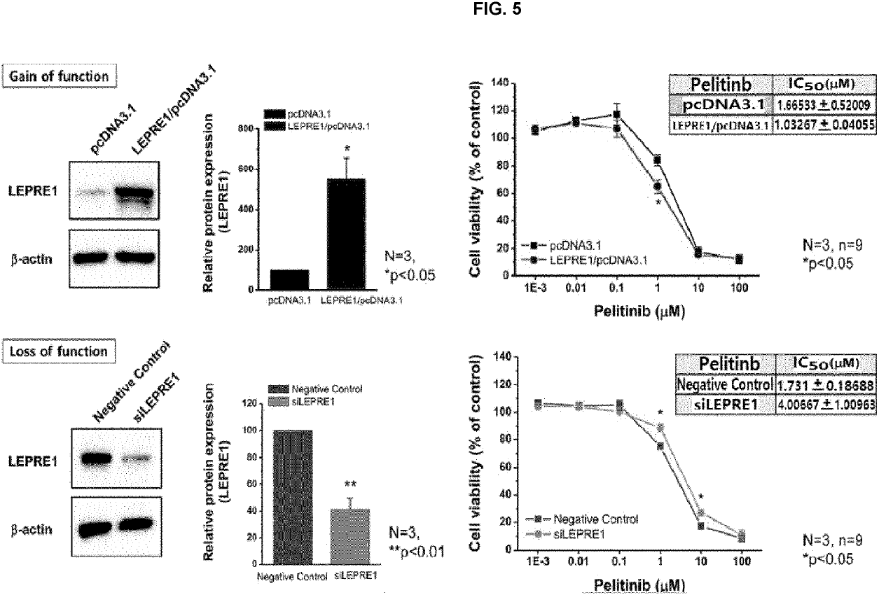

As illustrated in FIG. 5, the overexpression and inhibited expression of the LEPRE1 gene were induced in the A549 cell line, followed by treatment with a peletinib drug, and Ic50 values of the gene were marked. The expression level of LEPRE1 was identified by Western blot, and a test (WST-1 assay) of drug responsiveness to pelitinib was carried out. Similar to the values predicted through GBLscan, as a result of overexpressing pcDNA3.1, LEPRE1/pcDNA3.1 in A549 in A549, and then treating A549 with pelitinib, IC50 values were shown as 1.66533±0.52009 and 1.03267±0.04055, respectively. It was confirmed that LEPRE1 overexpression had the effect of increasing drug sensitivity to pelitinib. As a result of transfecting A549 with a negative control and LEPRE1 siRNA (si2293), and then treating A549 with pelitinib, IC50 values were shown as 1.731±0.18688 and 4.0067±1.00963, respectively. It was confirmed that the inhibited LEPRE1 expression had the effect of reducing drug sensitivity to pelitinib.

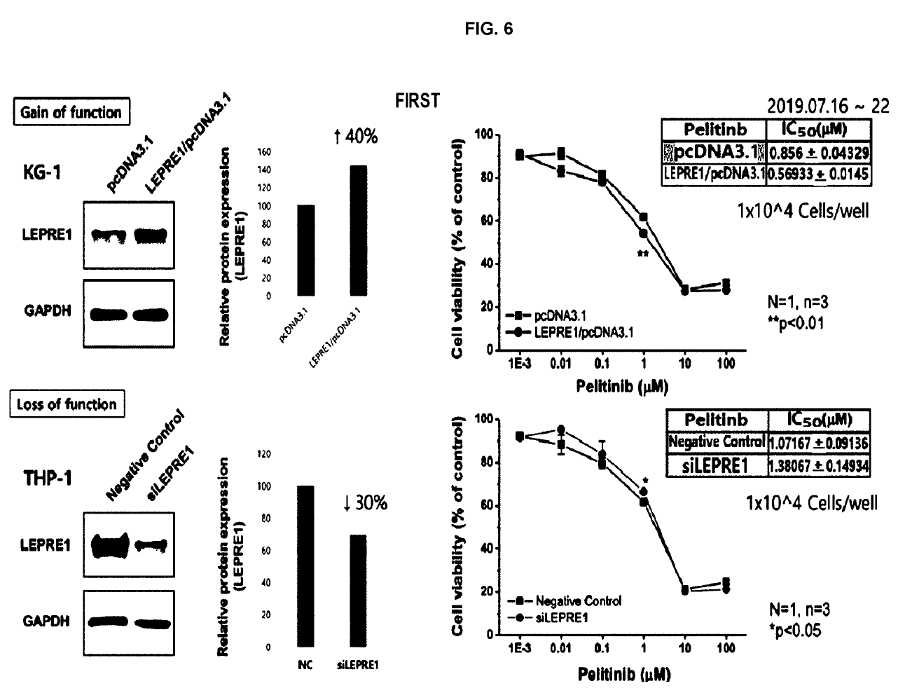

As illustrated in FIG. 6, subsequently, the overexpression and inhibited expression of the gene in hematological cancer cell lines KG-1 and THP-1 succeeded with electroporation, and sequential drug treatment was performed to verify changes in drug responsiveness. First to third experiments were consecutively carried out, and the first experiment succeeded in creating an expression level of about 140% upon overexpression, and an expression level of 70% upon inhibited expression. In the case of drug responsiveness, drug sensitivity was shown upon overexpression, and drug resistance was shown upon inhibited expression, as compared to cells showing normal expression levels.

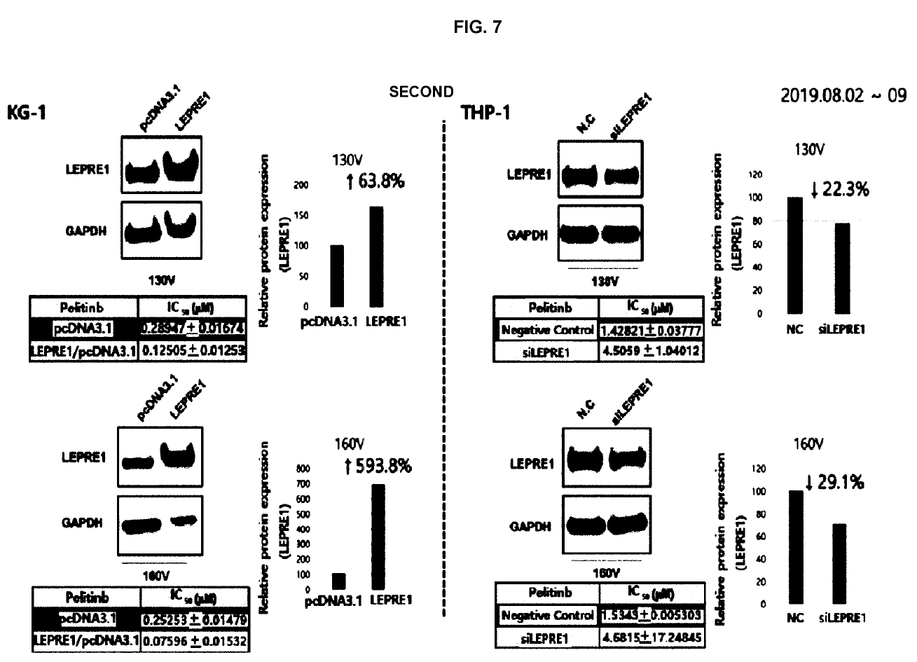

As illustrated in FIG. 7, conditions for the overexpression and inhibited expression of the gene through electroporation were specified, and voltage and time were adjusted, to repeat the experiments for overexpression and inhibited expression. Conditions for a significant increase or decrease in the expression level of the gene in KG-1 and THP-1 cell lines were found to examine drug responsiveness of the cells. In addition, the repeatability of the experiments was verified by obtaining the same result in the repeated experiments.

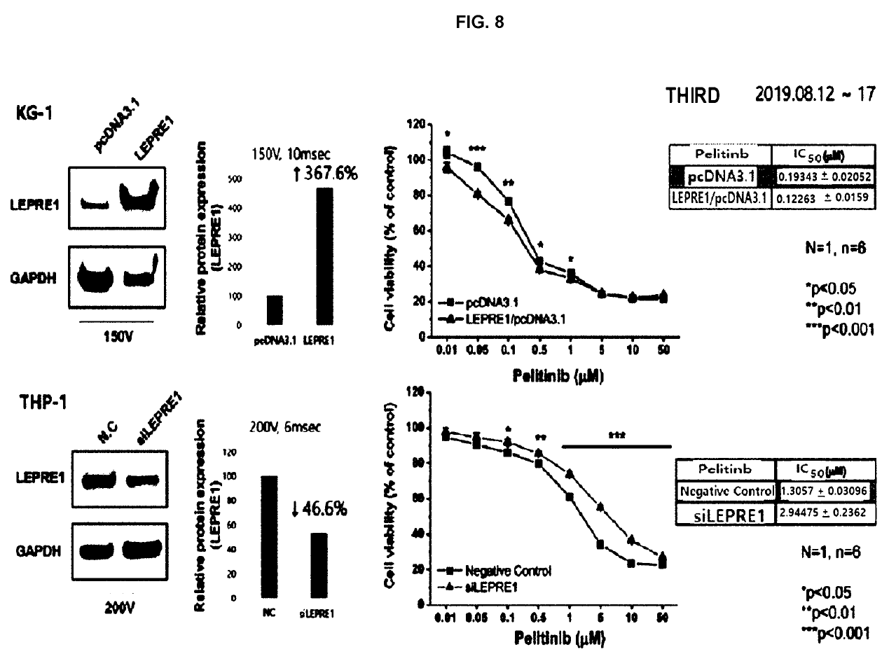

As illustrated in FIG. 8, the voltage and time conditions under which the inhibited expression and overexpression of the LEPRE1 gene in two hematological cancer cell lines, KG-1 and THP-1, are properly performed were found to create conditions for the overexpression and inhibited expression of the gene. Subsequently, the cells were treated with a peletinib drug, and the drug responsiveness of cells in which the gene was overexpressed and cells in which the expression of the gene was inhibited was measured and compared with the predicted values of GBLscan.

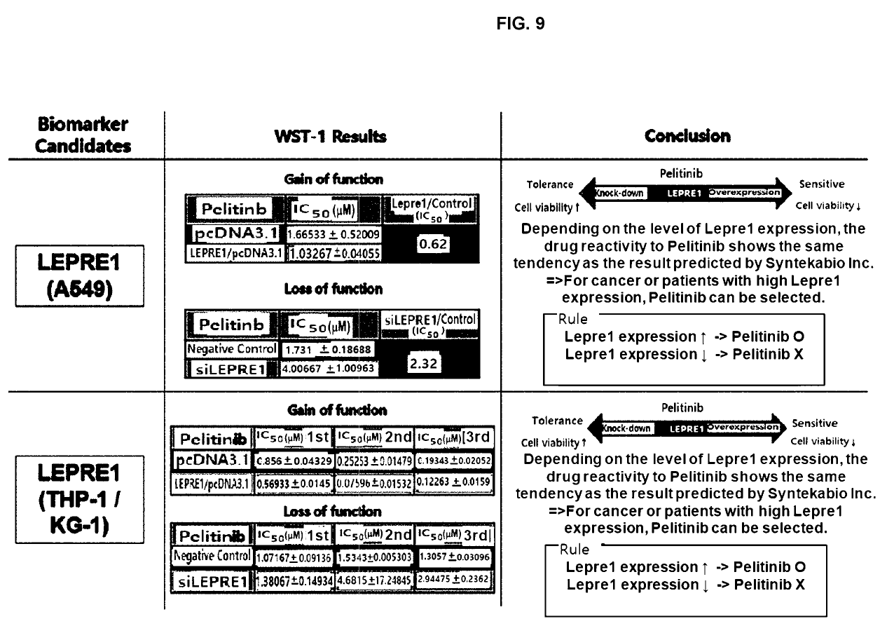

As illustrated in FIG. 9, as a result of transfecting A549 with a negative control and LEPRE1 siRNA (si2293), and then treating the same with pelitinib, IC50 values were shown as 1.731±0.18688 and 4.0067±1.00963, respectively. The loss-of-function effect whereby drug sensitivity to pelitinib decreases was confirmed when LEPRE1 expression was inhibited. This result is the same as the predicted result based on the drug responsiveness of about 1,000 cell lines to 263 drugs in A549 cells.

1. A method to determine a sensitivity or resistance to a drug comprising:
measuring an expression level of a gene in a cancer cell line; and
determine the sensitivity is high based on overexpression of the gene, and the resistance is high based on underexpression of the gene.

2. The method of claim 1, wherein the drug is an EGFR inhibitor.

3. The method of claim 1, wherein the drug is Pelitinib.

4. The method of claim 1, wherein the drug has multi-target efficacy as shown in Table 4 or an efficacy profile similar thereto.

5. The method of claim 1, wherein the gene is LEPRE1 gene.

6. The method of claim 1, wherein the gene is selected from the group consisting of genes listed in Table 2 that regulate the extracellular matrix environment.

7. The method of claim 1, wherein the gene is selected from the group consisting of genes listed in Table 3 that regulate the metabolism of collagen.

8. The method of claim 1, wherein the cancer cell line is selected from the group consisting of a hematological cancer cell line and a lung cancer cell line.

9. The method of claim 1, wherein the cancer cell line is selected from the group consisting of THP-1, KG-1, and A549.

10. The method of claims 1, wherein the gene is LEPRE1 gene and the expression level of the LEPRE1 gene is measured by detecting at least one alteration selected from Table 1.

11. A companion diagnostic composition for determining the sensitivity of an EGFR inhibitor drug, the companion diagnostic composition comprising an agent for measuring an RNA expression level of a LEPRE1 gene or an agent for specifying a protein expression level of the LEPRE1 gene.

12. The companion diagnostic composition of claim 11, wherein the agent for measuring an RNA expression level of a LEPRE1 gene is selected from the group consisting of a sense primer, an antisense primer and a probe that complementarily bind to the LEPRE1 gene or RNA thereof.

13. The companion diagnostic composition of claim 11, wherein the agent for specifying a protein expression level of the LEPRE1 gene is selected from the group consisting of an antibody, an aptamer and a probe that specifically binds to a protein encoded by the LEPRE1 gene.

14. The companion diagnostic composition of claims 11, wherein the companion diagnostic composition comprises at least one alteration selected from Table 1 below that predicts the expression level of the LEPRE1 gene.

15. A method of discovering a gene for determining drug sensitivity comprising:
(A) selecting a candidate gene for determining responsiveness to a target drug through cancer companion diagnostic marker scanning (GBLscan);
(B) realizing an overexpressed state of the candidate gene in a cell line targeted by the target drug;
(C) calculating responsiveness of the target drug to the targeted cell line, in the state in which the candidate gene is overexpressed, obtained in (B);
(D) realizing an underexpressed state of the candidate gene in the cell line targeted by the target drug;
(E) calculating responsiveness of the target drug to the targeted cell line, in the state in which the candidate gene is underexpressed, obtained in (D); and
(F) verifying whether or not the candidate gene is a marker for determining responsiveness to the target drug, compared with the responsiveness calculated in (C) and (E).

16. The method of claim 15, wherein, in (B), the overexpressed state of the candidate gene is realized using pcDNA3.1.

17. The method of claim 15, wherein, in (D), the underexpressed state of the candidate gene is realized using siRNA.

18. The method of claims 15, wherein, in (C) and (E), the responsiveness is calculated through IC50 values of the target drug for the targeted cell line.

19. The method of claim 18, wherein the target drug is an EGFR inhibitor drug.

20. The method of claim 15, wherein the candidate gene is a LEPRE1 gene.
